(19) United States
(12) Patent Application Publication
(10) Pub. No.: US 2017/0224772 A1
(43) Pub. Date: Aug. 10, 2017

(54) COMPOSITION AND KIT FOR PREVENTION OR TREATMENT OF ARTHRITIS, AND METHOD USING THE SAME

(71) Applicant: Samsung Electronics Co., Ltd., Suwon-si (KR)

(72) Inventor: Eun wha CHOI, Seoul (KR)

(73) Assignee: Samsung Electronics Co., Ltd., Suwon-si (KR)

(21) Appl. No.: 15/313,734

(22) PCT Filed: May 26, 2015
(86) PCT No.: PCT/KR2015/005241

§ 371 Date: Nov. 23, 2016

(30) Foreign Application Priority Data
May 27, 2014 (KR) 10-2014-0063827

(51) Int. Cl. A61K 38/17

(52) U.S. Cl. CPC A61K 38/1774; A61K 35/28

(57) ABSTRACT

Provided are a pharmaceutical composition and kit for prevention or treatment of arthritis, and a method of preventing or treating arthritis by using the same. The provided pharmaceutical composition, the kit, and the method have an excellent effect on the prevention and treatment of arthritis.

### TECHNICAL FIELD

The present disclosure relates to a composition and a kit for prevention or treatment of arthritis, and a method of preventing or treating arthritis by using the same.

### BACKGROUND ART

Arthritis, which is inflammation at a joint generated by a variety of causes, is accompanied by symptoms such as pain, edema, and fever. Among various types of arthritis, rheumatoid arthritis is a systemic chronic inflammatory disease and an autoimmune disease. Therapeutic agents of rheumatoid arthritis include nonsteroidal antiinflammatory drugs (NSAID), disease-modifying anti-rheumatic drugs (DMARD), adrenocortical hormones, and tumor necrosis factor (TNF) antagonists.

Activation of a T cell requires two signals provided by an antigen-presenting cell (APC). The first is a signal representing the recognition by a T cell receptor/CD3 complex that an antigen is provided by a major histocompatibility complex (MHC) of an APC. The second is a signal inducing the differentiation of a T cell, and the signal is also known as a costimulation signal of T cell CD28 and APC. Cytotoxic T-lymphocyte antigen 4 (CTLA4), which is also known as CD152, is a protein receptor that down-regulates the immune system and exists on the surface of a T cell. T cell activity is increased by stimulating a CD 28 receptor of a T cell, while the T cell activity is decreased by stimulating a CTLA4 receptor. Since the binding force between a CTLA4 receptor and B7 of an APC is greater than that between a CD 28 receptor and B7 of an APC, a CTLA4 receptor may block a costimulation signal of a T cell to discontinue T cell activity.

Abatacept, which is a fusion protein including an extracellular domain of CTLA4 and an Fc domain of immunoglobulin IgG1 (CTLA4Ig), is a rheumatoid arthritis therapeutic agent which is being clinically applied in the U.S. after acquiring FDA approval (Bristol-Myers Squibb; Brand name: Orencia). In Korea, abatacept has been approved to be used for monotherapy or combination therapy with a DMARD except a TNF antagonist in adult patients having moderate to severe active rheumatoid arthritis or in juvenile patients having idiopathic arthritis.

However, since the in vivo effective time of the CTLA4Ig fusion protein, represented by the in vivo half-life, is short (about 16.7 days in healthy individuals, about 13.1 days in rheumatoid arthritis patients, and about 14.3 days in adult rheumatoid arthritis patients in cases of hypodermic injection), the CTLA4Ig fusion protein should be continuously injected and costs are high. In addition, the CTLA4Ig fusion protein is not targeted to a joint.

Therefore, there is still a need for a pharmaceutical composition which may continuously express the CTLA4Ig fusion protein and be targeted to a joint which is the disease site of rheumatoid arthritis.

### DISCLOSURE OF INVENTION

### Technical Problem

Provided is a pharmaceutical composition for prevention or treatment of arthritis, wherein the pharmaceutical composition includes a mesenchymal stem cell expressing a fusion protein including a first polypeptide including cytotoxic T-lymphocyte antigen 4 (CTLA4) or a fragment thereof and a second polypeptide including an immunoglobulin constant region.

Provided is a kit for prevention or treatment of arthritis, wherein the kit includes an expression vector including a first polynucleotide encoding CTLA4 or a fragment thereof and a second polynucleotide encoding an immunoglobulin constant region; and a mesenchymal stem cell.

Provided is a method of preventing or treating arthritis by using the pharmaceutical composition for prevention or treatment of arthritis.

### Solution to Problem

This application claims the benefit of Korean Patent Application No. 10-2014-0063827, filed on May 27, 2014, in the Korean Intellectual Property Office, the disclosure of which is incorporated herein in its entirety by reference.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Provided is a pharmaceutical composition for prevention or treatment of arthritis, wherein the pharmaceutical composition includes a mesenchymal stem cell expressing a fusion protein including a first polypeptide including cytotoxic T-lymphocyte antigen 4 (CTLA4) or a fragment thereof and a second polypeptide including an immunoglobulin constant region.

CTLA4, which is also known as CD152, is a protein receptor that down-regulates the immune system and exists on the surface of a T cell. CTLA4 is similar to CD28 that is a costimulatory protein of a T cell. Both the T cell CTLA4 and CD28 are bound to CD80 (also known as B7-1) and CD86 (also known as B7-2) of an antigen presenting cell (APC). While CD28 transmits a stimulatory signal to a T cell, CTLA4 transmits an inhibitory signal. CTLA4 includes an extracellular domain, a transmembrane domain, and an intracellular tail. CTLA4 may be a polypeptide having an amino sequence of GenBank Accession No. NP_001032720 (human CTLA4) or NP-033973 (mouse CTLA4), or a polypeptide encoded by a polynucleotide sequence of GenBank Accession No. NM_001037631 (human CTLA4) or NM-009843 (mouse CTLA4).

The CTLA4 or a fragment thereof may include a CTLA4 extracellular domain. A CTLA4 fragment may be a CTLA4 part including a CTLA4 extracellular domain. The CTLA4 extracellular domain may be encoded by a polynucleotide sequence of SEQ ID NO: 2. The CTLA4 of a fragment thereof may a wild type isolated in vivo from a mammal such as human, dog, cat, goat, pig, mouse, rabbit, hamster, rat, and guinea pig, a recombinant type obtained from a transformed animal cell or microorganism, or a derivative thereof, or a gene synthesis product.

An immunoglobulin constant region is a region excluding an Fab (fragment, antigen binding) region from an immunoglobulin of a full-length antibody. The immunoglobulin constant region may be a wild type isolated in vivo from a mammal such as human, dog, cat, goat, pig, mouse, rabbit, hamster, rat, and guinea pig, a recombinant type obtained from a transformed animal cell or microorganism, or a derivative thereof, or a gene synthesis product. An immunoglobulin constant region may be a constant region of IgG, IgA, IgM, IgE, or IgD, or a combination thereof. An immunoglobulin constant region may include an Fc (fragment, crystallizable) region. An Fc region may include a heavy chain constant region 2 (CH2) and a heavy chain constant region 3 (CH3), and a hinge region in the heavy chain constant regions. The immunoglobulin constant region may be IgG1, IgG2, IgG3, or IgG4, or a combination thereof. The heavy chain constant region may include a gamma (γ), mu (μ), alpha (α), delta (δ), or epsilon (Ε) type constant region, more specifically, a γ1, γ2, γ3, γ4, α1, or α2 constant region as a subclass constant region. The light chain constant region may include a kappa or lambda type constant region. For example, the immunoglobulin constant region may be an immunoglobulin γ1 constant region. For example, the immunoglobulin constant region may be encoded by a polynucleotide sequence of SEQ ID NO: 3.

The term “mesenchymal stem cell (MSC)” used herein refers to a multipotent stromal cell that can be differentiate into various cell types including osteoblast, chondrocyte, and adipocyte. An MSC may differentiate into cells of specific organs such as bone, cartilage, fat, tendon, nerve tissue, fibroblast, and muscle cell. An MSC may be separated or purified from adipose tissue, bone marrow, peripheral nerve blood, cord blood, periosteum, dermis, or a tissue from a mesoderm. An MSC may be separated from an adipose tissue, or cultured from a cell separated from an adipose tissue. For example, an MSC may be a human MSC.

The fusion protein including a first polypeptide including CTLA4 or a fragment thereof and a second polypeptide including an immunoglobulin constant region may be a protein combined with the first polypeptide and the second polypeptide at an N-terminal of the fusion protein.

The fusion protein may be secreted from an MSC. For the secretion from an MSC, the fusion protein may be further fused with a signal peptide at an N-terminal of the fusion protein. The term “signal peptide” used herein is also called signal sequence, leader sequence, or leader peptide. In a protein translation process, a precursor polypeptide is combined with an endoplasmic reticulum membrane to form a membrane bound polysome, which synthesizes a polypeptide chain to pass through the endoplasmic reticulum membrane. Immediately after passing through the endoplasmic reticulum membrane, a signal peptide hydrolase included in an endoplasmic reticulum inner membrane cleaves and eliminates a signal peptide, and the synthesized polypeptide exists in the inner membrane of an endoplasmic reticulum. Then, the synthesized polypeptide is secreted extracellularly by exocytosis. For example, a signal peptide may be oncostatin M signal peptide. For example, a signal peptide may be encoded by a polynucleotide sequence of SEQ ID NO: 1.

The arthritis may be osteoarthritis, rheumatoid arthritis, psoriatic arthritis, gout, septic arthritis, juvenile idiopathic arthritis, or lupus.

The term “prevention” used herein refers to all of the actions in which a disease is restrained or the occurrence of a disease is retarded by the administration of the pharmaceutical composition. The term “treatment” used herein refers to all of the actions in which a disease takes a turn for the better or is modified favorably by the administration of the pharmaceutical composition.

The pharmaceutical composition may further include a pharmaceutically allowable additive, and may be formulated as a unit administration formulation suitable for administering to a patient by conventional methods in the pharmaceutical field. For example, the formulation may be a parenteral formulation for injection or local administration. For example, the composition may be used parenterally as an injection of an aseptic solution or a suspension prepared by using water, saline solution, or other pharmaceutically allowable solvents. For example, the composition may be formulated by mixing with a pharmaceutically allowable carrier or a medium, for example, sterile water, saline solution, vegetable oil, an emulsifying agent, a suspending agent, a surfactant, a stabilizer, an excipient, a vehicle, a preservative, or a binder into a pharmaceutically acceptable unit dosage type.

The pharmaceutical composition may be administered parenterally. The parenteral administration may be intradermal administration, subcutaneous administration, intra-muscular administration, or intravenous administration.

The MSCs of the pharmaceutical composition may be cryopreserved, and may be frozen or thawed by a method known in this art pertaining to the present invention. The MSCs may be stored in a cell preservation solution. For example, the MSCs may be stored in a composition including fetal bovine serum (FBS), dimethylsulfoxide (DMSO), or a combination thereof, but a cell preservation solution is not limited thereto. The composition may include, for example, from about 1×10⁶ to about 1×10⁷ cells in 1 mL, but the number of cells included in the composition may be different according to relevant conditions.

The pharmaceutical composition may be administered in a pharmaceutically effective amount. The term “pharmaceutically effective amount” used herein refers to an amount that is sufficient to treat a disease. An effective dosage level may be determined according to factors including severity of disease, patient's age, weight, health conditions, sex, sensitivity to drug, drug administration time, administration route, discharge rate, treatment period, and drugs which are mixed or used in combination with the composition of the present invention, and other factors which are well known in the medical field. The dosage of MSCs varies in a wide range and is determined by individual requirements in each specific case. For example, in the case of parenteral administration, the daily dosage of the MSCs may be from about 1×10⁴ to about 1×10⁷ cells/kg weight, and the composition may be administered once or several times a day.

The pharmaceutical composition may be administered sequentially or simultaneously with other conventional arthritis therapeutic agents.

Provided is a kit for prevention or treatment of arthritis, wherein the kit includes an expression vector including a first polynucleotide encoding CTLA4 or a fragment thereof and a second polynucleotide encoding an immunoglobulin constant region; and a mesenchymal stem cell.

CTLA4 or a fragment thereof, an immunoglobulin constant region, MSC, arthritis, prevention, and treatment are described above.

An expression vector refers to a vector capable of expressing an introduced gene in a cell. For example, an expression vector may be adenovirus vector, retrovirus vector, adeno-associated virus vector, herpes simplex virus vector, SV40 vector, polyoma virus vector, papilloma virus vector, picarno virus vector, vaccinia virus vector, or lenti virus vector. In addition, an expression vector may be a non-viral vector such as minicircule DNA vector and peptide vector, but is not limited thereto.

The expression vector includes a first polynucleotide encoding CTLA4 or a fragment thereof and a second polynucleotide encoding an immunoglobulin constant region. The first polynucleotide may include a polynucleotide of SEQ ID NO: 2. The second polynucleotide may include a polynucleotide of SEQ ID NO: 3. The expression vector may further include a polynucleotide of SEQ ID NO: 1.

The kit may further include a transfection reagent that is needed to introduce the expression vector to an MSC. For example, a transfection reagent may be lipofectamin.

Provided is a method of preventing or treating arthritis, wherein the method includes administrating an MSC expressing a fusion protein including a first polypeptide including CTLA4 or a fragment thereof and a second polypeptide including an immunoglobulin constant region to a subject.

CTLA4 or a fragment thereof, an immunoglobulin constant region, MSC, arthritis, prevention, and treatment are described above.

The MSCs may be administered parenterally. The parenteral administration may be intradermal administration, subcutaneous administration, intramuscular administration, intra-articular administration or intravenous administration. An administration amount may be determined according to factors including severity of disease, patient's age, weight, health conditions, sex, sensitivity to drug, drug administration time, administration route, discharge rate, treatment period, and drugs which are mixed or used in combination with the composition of the present invention, and other factors which are well known in the medical field. The dosage of MSCs varies in a wide range and is determined by individual requirements in each specific case. For example, in the case of parenteral administration, the daily dosage of the MSCs may be from about 1×10⁴ to about 1×10⁷ cells/kg weight, and the composition may be administered once or several times a day.

The subject may be one mammal selected from the group consisting of human, dog, cat, pig, horse, cattle, sheep, mouse, and monkey.

The arthritis may be rheumatoid arthritis.

### BRIEF DESCRIPTION OF DRAWINGS

These and/or other aspects will become apparent and more readily appreciated from the following description of the embodiments, taken in conjunction with the accompanying drawings in which:

FIG. 1a is a schematic diagram of a murine CTLA4Ig-pLenti6/V5 TOPO expression plasmid, and FIG. 1b is an image of the transduced human adipose tissue-derived mesenchymal stem cells (hASCs);

FIG. 2 is a graph showing the arthritis severity score according to the number of days following immunization;

FIGS. 3a to 3c are the graphs showing the concentration of anti-mouse type II collagen antibody in serum, the concentration of C-telopeptide I in serum, and the concentration of C-telopeptide II in serum in each group;

FIG. 4a is an image of a knee joint tissue stained by using H&E or Safranin O, FIG. 4b is a graph showing the severity of cartilage injury in a score from 0 points (No injury) to 4 points (Severe injury), and FIG. 4c is a graph showing the severity of morphological defects in a score from 0 points (No defect) to 4 points (Severe defect); and

FIG. 5 is an image of mice tissues obtained by administering fluorescence-labeled stem cells to tail veins of mice.

### MODE FOR THE INVENTION

Hereinafter, the present invention will be described in further detail with reference to examples. However, these examples are illustrative purposes only and are not to be construed to limit the scope of the present invention.

### EXAMPLE

### Verification of Rheumatoid Arthritis Prevention or Treatment Effect of MSCs Expressing CTLA4

1. Preparation of MSCs Expressing CTLA4Ig

Under the agreement of a donor, human abdominal subcutaneous adipose tissue was obtained by liposuction. Human adipose tissue-derived mesenchymal stem cells (hASCs) were separated from adipose tissue vascular fraction and cultured under Good Manufacturing Practice conditions. Under the test approved by the Institutional Review Board (IRB) of Samsung Medical Center, hASCs prepared by removing personal identification information were used.

A polynucleotide sequence encoding an extracellular domain of a mouse CTLA4 protein as a target protein and a polynucleotide encoding a hinge-CH2-CH3 domain of a murine immunoglobulin G1 (IgG1) were used. An oncostatin signal sequence was used so that a target gene may be secreted at a target region.

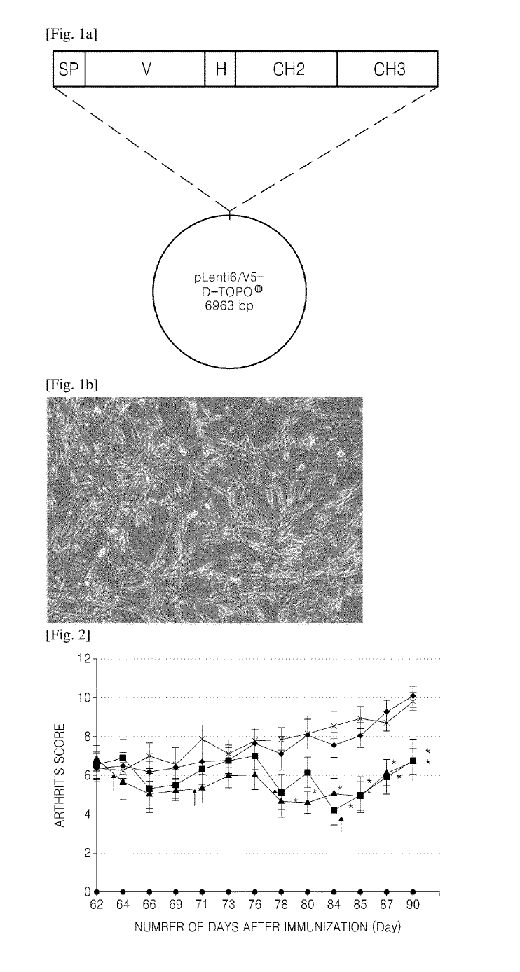

Specifically, from a 5′-terminal, a human oncostatin M signal sequence (SEQ ID NO: 1 which is from the 53rd to the 127th nucleotides of GenBank Accession No. NM_020530.3), a polynucleotide encoding the extracellular domain of murine CTLA4 (SEQ ID NO: 2 which is from the 258th to the 629th nucleotides of GenBank Accession No. NM_009843.4), and a polynucleotide encoding the hinge-CH2-CH3 domain of the murine immunoglobulin gamma 1 constant region (SEQ ID NO: 3 which is from the 772th to the 1452th nucleotides of GenBank Accession No. AB097849) were fused. The fused genes were cloned into pLenti6/V5-D-TOPO® (Life Technologies) to prepare a murine CTLA4Ig-pLenti6/V5 TOPO expression plasmid. The human oncostatin M signal sequence was a signal sequence secreting the target protein into body fluid. The CTLA4 was to inhibit a B7:CD28 co-stimulatory signal, and the hinge-CH2-CH3 domain of the IgA was to extend in vivo the half-life of the target protein. FIG. 1a is a schematic diagram of a murine CTLA4Ig-pLenti6/V5 TOPO expression plasmid (SP: signal sequence, V: polynucleotide sequence encoding the extracellular domain of murine CTLA4, H: polynucleotide sequence encoding a hinge region, CH2: polynucleotide sequence encoding CH2 region, and CH3: polynucleotide sequence encoding CH3 region).

The introduction of the CTLA4Ig fusion gene that was the target gene to MSCs was performed by using ViraPower™ Lentiviral Expression System (Life Technologies). Co-transduction of a ViraPower packaging mix and the murine CTLA4Ig-pLenti6/V5 TOPO expression plasmid prepared above was performed into 293 FT cell line (Life Technologies) by using lipofectamine° 2000 (Life Technologies). Virus particles were obtained and transduced into the prepared hASCs. FIG. 1b is an image of the transduced hASCs.

5 μg/ml of blasticidin (Life Technologies) was used to select the cell line to which the murine CTLA4Ig fusion gene was introduced. To verify the expression and extra-cellular secretion of CTLA4Ig by the selected cells, the supernatant of the cell culturing medium was collected, and the concentration of the murine CTLA4Ig fusion protein in the supernatant was determined by enzyme-linked enzyme-linked immunospecific assay (ELISA) by using CTLA4 DuoSet® ELISA Development kit (R&D systems, Cat. No. DY476). Then, CTLA4Ig-hASCs were selected.

1-2. Preparation of Rheumatoid Arthritis Animal Model and Administration of MSCs Expressing CTLA4Ig

Chicken type II collagen (Sigma-Aldrich) was mixed with 0.05 M acetic acid at 4° C. to the concentration of 2 mg/ml. The resulting mixture and Freund's complete adjuvant (CFA) (Sigma-Aldrich) were mixed at an equal amount ratio, and then the resulting mixture was homogenized at 30000 rpm for three minutes by using a homogenizer (Polytron PT3100D) to prepare an antigen material for administration. For a first immunization, the antigen material for administration was intradermally injected to a mouse tail (DBA/1 mouse, six weeks old) (Day 1). Three weeks later, the CFA was replaced by Freund's incomplete adjuvant (IFA) (Sigma-Aldrich), and boosting was performed by the same method.

Mice were divided into five groups, and saline solution, hASCs, and CTLA4Ig-hASC were intravenously injected to mouse tails on Day 63, Day 70, Day 77, and Day 84 after immunization as shown in Table 1 below. To compare the effect on rheumatoid arthritis, 1 mg/kg body weight of methotrexate (MTX) (Medac), which is used as a therapeutic agent for rheumatoid arthritis, was intraperitoneally injected a total of 12 times at a frequency of three times per week.

TABLE 1

| | | Administered material, administration |
|---|---|---|
| | | method, and number |
| Group | Animal model | of times of administration |
| Normal group (n = 10) | Normal mice in which | 150 μl of saline solution, tail |
| | rheumatoid arthritis was | vein administration, a total of |
| | not induced. | four times |
| Negative control group | Mice in which rheumatoid | 150 μl of saline solution, tail |
| (n = 13) | arthritis was induced. | vein administration, a total of |
| | | four times |
| hASC group (n = 12) | | 2 × 10⁶ hASCs/150 μl of |
| | | Dulbecco's Phosphate Buffered |
| | | Saline (dPBS), tail vein administration, |
| | | a total of four times |
| CTLA4Ig-hASC group | | 2 × 10⁶ CTLA4Ig-hASC/150 μl |
| (n = 13) | | of dPBS, tail vein administration, |
| | | a total of four times |
| MTX group (n = 7) | | 1 mg/kg body weight, a total of |
| | | 12 times at a frequency of three |
| | | times per week, intraperitoneal |
| | | administration |

One week after the last transplantation, all the animals were sacrificed to obtain blood and tissue samples.

1-3. Assessment of Arthritis Severity

In the mice of Example 1-2, from Day 62 after the immunization, the severity of arthritis at the fore paw and the hind paw was assessed three times per week in a score from 0 point (No arthritis) to 4 point (Severe arthritis) according to the degree of edema and flare. The assessment was performed at each leg with the maximum possible score of 16. The clinical score was measured at each mouse by Day 90 after the immunization. The data obtained from each group were compared by performing a one-way analysis of variance (ANOVA) and then Tukey's multiple comparison tests.

FIG. 2 is a graph showing the arthritis severity score according to the number of days following immunization (: Normal group, ♦: Negative control group, ▪: hASC group, ▴: CTLA4Ig-hASC group, ×: MTX group). In FIG. 2, the time when saline solution, hASC, or CTLA4Ig-hASC was administered was marked as “↑” and a significant difference in comparison with the negative control group was marked as “*” (p<0.05).

As shown in FIG. 2, the arthritis severity score of the hASC group and the CTLA4Ig-hASC group was significantly decreased after three times of administration in comparison with that of the negative control group. The arthritis severity score of the MTX group showed a pattern that was similar to that of the negative control group.

1-4. Detection of Anti-Type II Collagen Antibody, C-Terminal Telopeptide of Type I Collagen, and C-Terminal Telopeptide of Type II Collagen in Serum

In the serum obtained in Example 1-2, anti-mouse type II collagen antibody, C-terminal telopeptide of Type I collagen (C-telopeptide I) that was a Type I collagen discharged during osteoclastic bone resorption, and C-terminal telopeptide of Type II collagen (C-telopeptide II) that was a chondrolysis product were detected by ELISA.

Specifically, anti-mouse type II collagen antibody, C-telopeptide I, and C-telopeptide II in the serum were measured by using an anti-mouse type II antibody collagen ELISA kit (Astarte Biologics), RatLap™ EIA kit (Immunodiagnostic Systems Ltd.), and a type II collagen ELISA kit (CUSABIO), respectively. The measurement was performed by following the protocols provided by respective manufacturers.

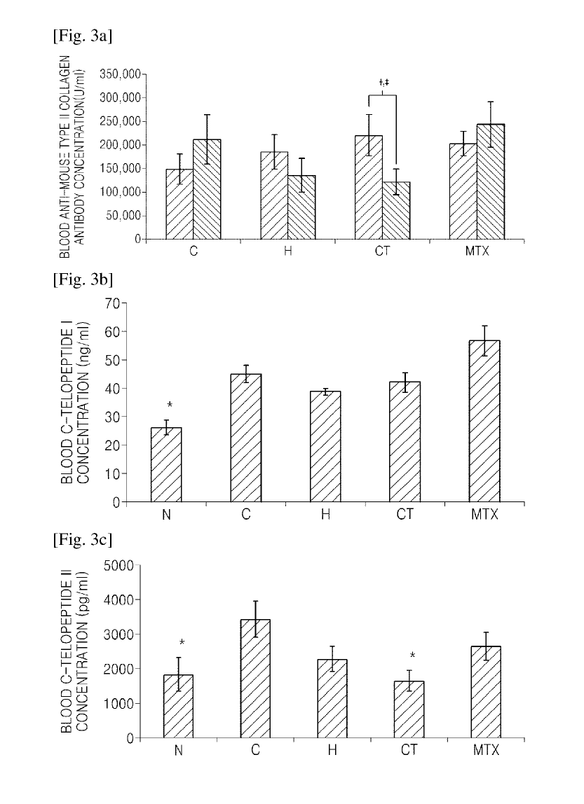

FIG. 3a is a graph showing the concentration of anti-mouse type II collagen antibody (U/ml) of each group ( [symbol] : before treatment, and [symbol] : after treatment) (C: Negative control group, H: hASC group, CT: CTLA4Ig-hASC group, and MTX: MTX group). The mean values of two related samples (before and after treatment) were compared by performing a paired t-test (†: p<0.05) and a Wilcoxon's signed rank test (‡: p<0.05). FIG. 3b and FIG. 3c respectively show the concentration of C-telopeptide I and C-telopeptide II in each group (N: Normal group, C: Negative control group, H: hASC group, CT: CTLA4Ig-hASC group, and MTX: MTX group). The concentration values were compared by performing one-way ANOVA and then Tukey's multiple comparison tests. A significant difference in comparison with the negative control group was marked as “*” (p<0.05).

As shown in FIG. 3a, the concentration of the anti-mouse type II collagen autoantibody in the serum after the treatment was significantly lower than that before the treatment in the CTLA4Ig-hASC group. In addition, as shown in FIG. 3b and FIG. 3c, the concentration of C-telopeptide I in the serum was not significantly different among the negative control group, the hASC group, the CTLA4Ig-hASC group, and the MTX group. However, the concentration of C-telopeptide II in the serum was significantly lower in the normal group and the CTLA4Ig-hASC group than in the negative control group.

1-5. Flowcytometric Analysis of Ratios of Regulatory T Cells and CD138 Cells

To verify the ratios of regulatory T cells and CD138 cells (plasma cells or B cell precursors) in the murine splenocytes obtained in Example 1-2, CD4, CD25 and Foxp3 that were markers of regulatory T cell, and CD138 that was a marker of CD138 cell were used. Plasma cells that are generated in an autoimmune reaction process produce autoimmune antibodies, and a humoral immunity reaction related with B cells is necessary in the progress of a chronic autoimmune disease.

A flowcytomety of murine splenocytes was performed by using fluorescein isothiocyanate (FITC) rat anti-mouse CD4 antibody (BD Biosciences), allophycocyanin (APC) rat anti-mouse CD25 antibody (BD Biosciences), phycoerythrin (PE) anti-mouse Foxp3 antibody (BD Biosciences), and PE rat anti-mouse CD138 antibody (BD Biosciences).

The ratio of CD4+Foxp3+cells in the normal group, the negative control group, the hASC group, the CTLA4Ig-hASC group, and the MTX group was 5.7±1.1%, 3.4±0.4%, 3.4±0.3%, 6.3±4.6%, and 4.4±3.3%, respectively. The ratio of CD138 cells in the normal group, the negative control group, the hASC group, the CTLA4Ig-hASC group, and the MTX group was 13.8±3.0%, 18.8±1.9%, 7.2±0.6%, 7.7±0.6%, and 17.2±2.4%, respectively. Therefore, there was not a significant difference in the regulatory T cell proliferation in the murine splenocytes between the control group and the treatment groups. However, the ratio of CD138 cells in the hASC group and the CTLA4Ig-hASC group was significantly lower than that of the negative control group.

1-6 Measurement of Concentration of Cytokine in Serum

To verify the variation of cytokine expression according to transplantation of hASC or CTLA4Ig-hASC, the serum, knee extract, splenocytes, and lymph node cells obtained in Example 1-2 were prepared.

Splenocytes of each group were separated. 2.5×10⁵/well of splenocytes cultured in 96-well plate (Nunc) in a final volume of 200 μl including or not including 100 μg/ml of Type II collagen (Sigma Aldrich), 2.5 μg/ml of concanavalin A (ConA) (Sigma Aldrich), and 2.5 μg/ml of lipopolysaccharide (LPS) (Sigma Aldrich). The cells were incubated in humidified air at 37° C. for 48 hours. Then, the supernatant of the splenocyte culture medium was obtained and stored at −70° C. By the same method, lymph node cells were cultured in a culture medium including ConA, and the supernatant of lymph node cell culture solution was obtained.

The cytokine concentration in serum, knee extract, and the obtained supernatant of the culture solutions of splenocytes and lymph node cells was measured by using a multiplex cytokine/chemokine kit (Milliplex MAP mouse cytokine/chemokine kit, Millipore) by following the protocol provided by the manufacturer. As cytokines, tumor necrosis factor (TNF)-αinterferon (IFN)-α, interleukin (IL)-1α, IL-1β, IL-2, IL-4, IL-6, IL-10, IL-12p70, IL-15, IL-17, keratinocyte-derived chemokine (KC), monocyte chemotactic protein (MCP)-1, macrophage inflammatory protein (MIP)-2, and regulated-upon-activation normal T-cell expressed and secreted (RANTES) were measured.

The cytokine concentration (pg/ml) in the serum and knee extract is shown in Tables 2 and 3, respectively. The cytokine concentration (pg/ml) in the splenocyte culture medium that was cultured in the presence of Type II collagen, ConA, an LPS is shown in Tables 4 to 6, respectively. The cytokine concentration (pg/ml) in the lymph node cell culture solution that was cultured in the presence of ConA is shown in Table 7.

The data obtained from each group was compared by performing a Kruskal Wallis test (†: P<0.05) or a (Mann-Whitney U test) (*: significant difference in comparison with the negative control group (p<0.05)).

TABLE 2

| | | Negative | | | |
|---|---|---|---|---|---|
| | Normal | control | | CTLA4Ig-hASC | |
| Serum | group | group | hASC group | group | MTX group |
| IL-6^{†} | 6.08 ± 1.80* | 117.00 ± 54.69 | 45.46 ± 19.75 | 27.41 ± 9.00 | 161.10 ± 59.69 |
| TNF-α^{†} | 4.76 ± 0.70* | 10.13 ± 0.96 | 8.16 ± 0.76 | 7.97 ± 0.88 | 5.22 ± 1.37 |
| IL-12p70^{†} | 3.05 ± 1.38 | 123.46 ± 127.26 | 22.62 ± 17.34 | Not | Not |
| | | | | detected* | detected* |
| MIP-2^{†} | 98.03 ± 8.50 | 104.06 ± 7.72 | 85.80 ± 14.52 | 70.79 ± 14.33* | 17.36 ± 8.79* |

TABLE 3

| | | Negative | | | |
|---|---|---|---|---|---|
| | Normal | control | | CTLA4Ig-hASC | |
| Knee extract | group | group | hASC group | group | MTX group |
| IL-1β^{†} | 7.16 ± 1.93 | 8.61 ± 2.17 | 2.98 ± 1.46 | 1.72 ± 0.84* | 4.54 ± 2.88 |
| IL-6 | 2.72 ± 0.41 | 16.42 ± 5.88 | 11.46 ± 6.78 | 5.64 ± 3.50 | 19.37 ± 11.87 |
| KC^{†} | 23.66 ± 2.44* | 260.33 ± 100.9 | 127.21 ± 62.81 | 94.07 ± 42.98 | 305.82 ± 147.86 |
| MIP-2^{†} | 23.21 ± 2.57 | 70.17 ± 20.86 | 14.67 ± 7.81* | 5.00 ± 3.15* | 50.36 ± 19.78 |
| RANTES^{†} | 1.78 ± 0.10 | 1.81 ± 0.14 | 0.66 ± 0.12* | 0.28 ± 0.06* | 0.20 ± 0.07* |

| Spleen-Type II | Negative | | CTLA4Ig-hASC | |
|---|---|---|---|---|
| collagen | control group | hASC group | group | MTX group |
| IL-10^{†} | 4.19 ± 1.28 | 22.98 ± 4.99* | 14.41 ± 3.53* | 3.75 ± 0.89 |
| IL-17^{†} | 2.09 ± 1.01 | 1.37 ± 0.61 | 0.31 ± 0.15* | 4.73 ± 2.01 |
| MCP-1^{†} | 418.43 ± 114.45 | 197.63 ± 41.61 | 159.24 ± 40.98* | 591.43 ± 222.2 |

TABLE 5

| | Negative | | CTLA4Ig-hASC | |
|---|---|---|---|---|
| Spleen-ConA | control group | hASC group | group | MTX group |
| IL-10^{†} | 93.33 ± 14.66 | 458.89 ± 58.59* | 352.16 ± 42.3* | 80.76 ± 23.19 |
| IL-17 | 968.29 ± 148.82 | 582.81 ± 212.81 | 595.95 ± 134.07 | 1345.14 ± 590.38 |
| IL-1α | 23.00 ± 3.13 | 13.61 ± 3.74 | 13.92 ± 2.30 | 21.02 ± 3.77 |
| IL-6 | 282.31 ± 44.29 | 156.14 ± 16.09 | 182.71 ± 19.49 | 279.78 ± 71.12 |
| KC^{†} | 14.78 ± 1.53 | 6.80 ± 0.85* | 8.52 ± 1.51* | 10.78 ± 1.86 |
| MCP-1^{†} | 235.18 ± 46.06 | 124.30 ± 19.88 | 114.62 ± 23.29 | 549.99 ± 220.33 |

TABLE 6

| | Negative | | CTLA4Ig-hASC | |
|---|---|---|---|---|
| Spleen-LPS | control group | hASC group | group | MTX group |
| IL-1α | 98.45 ± 9.69 | 73.52 ± 8.27 | 77.31 ± 9.79 | 112.01 ± 27.82 |
| IL-2^{†} | 7.63 ± 1.47 | 22.47 ± 2.74* | 16.73 ± 3.33* | 11.02 ± 3.36 |
| IL-4† | 0.60 ± 0.19 | 3.12 ± 0.25* | 1.78 ± 0.37* | 0.37 ± 0.24 |
| IL-6† | 4396.93 ± 379.32 | 2933.08 ± 255.77* | 3190.74 ± 408.24* | 4676.79 ± 1097.10 |
| KC^{†} | 243.19 ± 19.28 | 143.47 ± 18.02* | 164.54 ± 24.14* | 199.78 ± 50.25 |
| MCP-1^{†} | 173.25 ± 22.77 | 72.76 ± 13.79* | 104.31 ± 21.29* | 250.79 ± 87.01 |
| MIP-2^{†} | 2313.30 ± 177.31 | 1430.13 ± 174.98* | 1591.59 ± 225.99* | 2022.27 ± 409.37 |
| RANTES^{†} | 148.05 ± 11.35 | 98.09 ± 7.65* | 107.45 ± 13.43* | 128.44 ± 24.46 |
| TNF-α^{†} | 105.96 ± 10.27 | 72.22 ± 9.25 | 78.06 ± 11.50 | 90.65 ± 20.41 |

TABLE 7

| | | Negative | | | |
|---|---|---|---|---|---|
| Lymph | Normal | control | | CTLA4Ig-hASC | |
| node-ConA | group | group | hASC group | group | MTX group |
| IL-10^{†} | 51.91 ± 23.04 | 35.19 ± 6.34 | 94.71 ± 36.96* | 62.04 ± 8.31* | 23.11 ± 4.08 |

As shown in Table 2, the concentrations of IL-12p70 and MIP-2 in serum were significantly lower in the CTLA4Ig-hASC group and the MTX group than in the negative control group. As shown in Table 3, in the knee extract, the RANTES concentration was significantly lower in the hASC group, the CTLA4Ig-hASC group, and the MTX group than in the negative control group; the MIP-2 concentration was significantly lower in the hASC group and the CTLA4Ig-hASC group than in the negative control group; and the IL-1β concentration was significantly lower in the CTLA4Ig-hASC group than in the negative control group. As shown in Table 4, in the culture medium of splenocytes which were stimulated by Type II collagen, the IL-10 concentration was significantly higher in the hASC group and the CTLA4Ig-hASC group than in the negative control group, and the IL-17 and MCP-1 concentrations were significantly lower in the CTLA4Ig-hASC group than in the negative control group. As shown in Table 5, the culture solution of splenocytes which were stimulated by ConA, the KC concentration was significantly lower in the hASC group and the CTLA4Ig-hASC group than in the negative control group, and the IL-10 concentration was significantly higher in the hASC group and the CTLA4Ig-hASC group than in the negative control group. In the hASC group and the CTLA4Ig-hASC group, the concentrations of IL-17, IL-1α, IL-6, and MCP-1 in serum showed a decreasing tendency. As shown in Table 6, in the culture medium of splenocytes which were stimulated by LPS, the concentrations of IL-6, KC, MCP-1, and RANTES were significantly lower in the hASC group and the CTLA4Ig-hASC group than in the negative control group, and the concentrations of IL-2 and IL-4 were significantly higher in the hASC group and the CTLA4Ig-hASC group than in the negative control group. However, the cytokine concentrations in the MTX group were not significantly different from those of the negative control group. As shown in Table 7, in the culture medium of lymph node cells which were simulated by ConA, the IL-10 concentration was significantly higher in the hASC group and the CTLA4Ig-hASC group than in the negative control group.

1-7. Histopathological Assessment and Verification of Morphologic Defects in Paw Joints

Knee cartilage tissues were stained to investigate the degree of cartilage injury and bone injury by rheumatoid arthritis, and micro-computed tomography images were obtained to verify morphologic defects in paw joints.

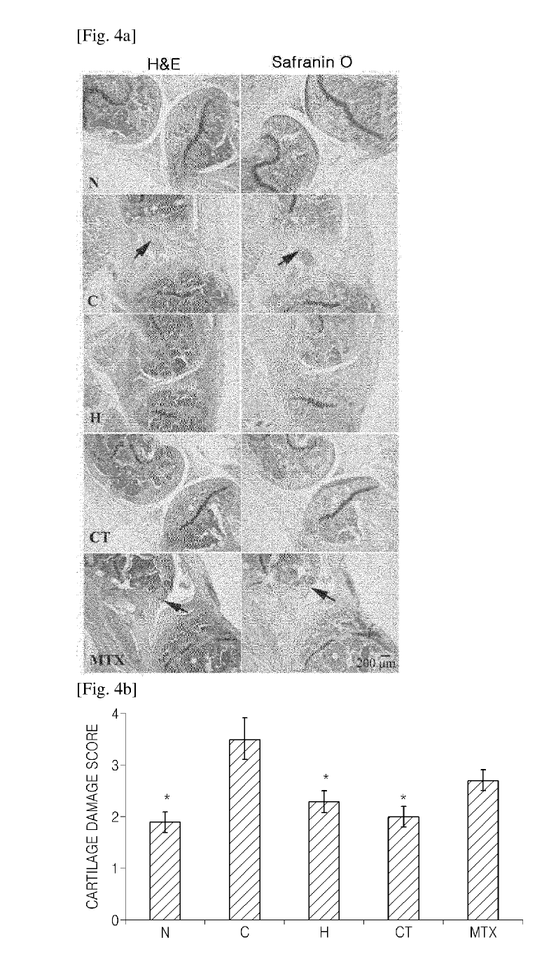

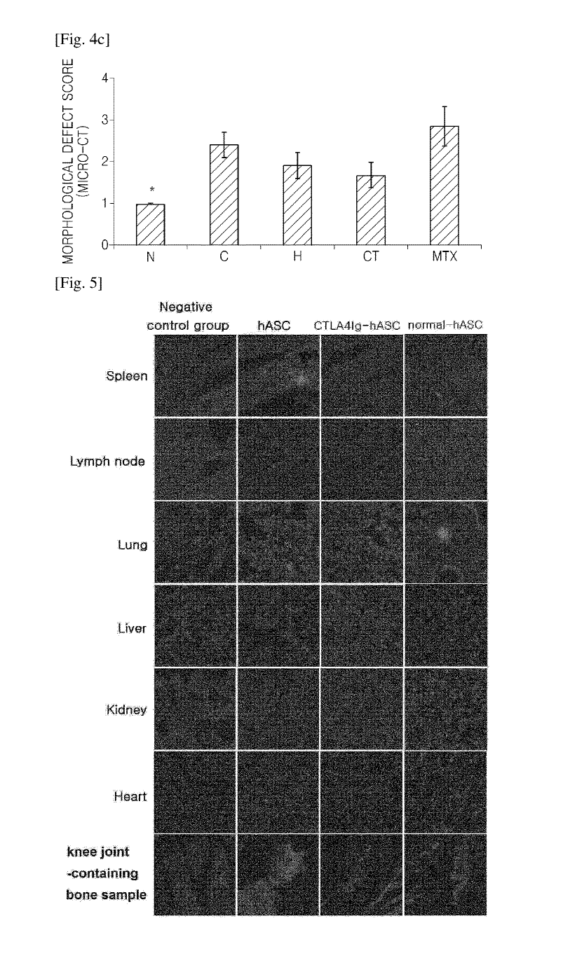

In Week 13 after the first immunization, knee cartilage tissues were obtained from the animals of Example 1-2. The obtained knee cartilage tissues were fixed by using 10% (v/v) neutral buffered formalin (Sigma-Aldrich), and the resulting solution was stirred by using a rapid decalcifier (Thermo Scientific) for 24 hours to decalcify. When an osseous tissue was easily cut by a razor, it was assumed that the tissue was completely decalcified. The decalcified osseous tissue was dehydrate and embedded in paraffin. To assess histopathological injury, the paraffin-embedded tissue was cut into 4 μm-thick slices, and the paraffin was removed from the slices by using xylene. Then, the slices were rehydrated by using a graded alcohol, and stained by using hematoxylin and eosin (H&E) (DAKO) and Safranin O (IHC World). The results of the staining are shown in FIGS. 4a and 4b. FIG. 4a is an image of a knee joint tissue stained by using H&E or Safranin O (magnified by 40 times, arrow: severe cartilage destruction), and FIG. 4b is a graph showing the severity of cartilage injury in a score from 0 point (No injury) to 4 points (Severe injury).

To verify morphological defects in paw joints, the mice paw obtained in Example 1-2 were fixed by using 4%(v/v) formalin (Sigma-Aldrich), and images of the mouse paws were obtained by using Inveon Preclinical CT (Siemens Healthcare) that is a micro-CT scanner at 40 μm of thickness, 0.6 second of exposure time, 70 keV of proton energy, and 400 μA of current. The images were reconstructed into three-dimensional images by using IRW software (Siemens Healthcare).

FIG. 4c is a graph showing the severity of morphological defects in a score from 0 point (No defect) to 4 point (Severe defect) (C: Negative control group, H: hASC group, CT: CTLA4Ig-hASC group, and MTX: MTX group). The data were compared among the groups by performing an ANOVA and then Tukey's multiple comparison tests. A significant difference in comparison with the negative control group was marked as “*” (p<0.05).

As shown in FIGS. 4a and 4b, the destruction of the joint cartilage was decreased in the hASC group and the CTLA4Ig-hASC group, while the joint cartilage was severely damaged in the negative control group and the MTX group. On the other hand, the results were consistent with the concentration of C-telopeptide II in serum. In addition, as shown in FIG. 4c, four mice out of the twelve mice in the negative control group, two mice out of the twelve mice in the hASC group, two mice out of the thirteen mice in the CTLA4Ig-hASC group, and four mice out of the seven mice in the MTX group showed a sever morphological defect in the paw joints. The result verified that the translpantation of CTLA4Ig-hASC had a cartilage-protecting effect.

1-8. Verification of In Vivo Distribution of Administered hASC

To verify the tissues in which the stem cells that had been administered to the mouse tail vein were distributed, 2×10⁶ hASCs or CTLA4Ig-hASCs which were labeled by using Cell Tracker™ CM-Dil (Life Technologies), which is a red fluorescence substance, according to the protocol provided by the manufacturer to the mice (n=3) in which rheumatoid arthritis was induced by the method described in Example 1-2 (A total of four times on Day 63, Day 70, Day 77, and Day 84 after the immunization).

An autopsy was performed with the mice one week after administering the CM-Dil-labeled stem cells to the mice (on the thirteenth week following the first immunization) to obtain the spleens, lymph nodes, lungs, livers, kidneys, hearts, and knee joints. The obtained samples were incubated by treating the sample with Tissue-Tek® optimum cutting temperature (O.C.T.) (Sakura Finetek) according to the protocol provided by the manufacturer, and then stored at −80° C.

The tissue slice samples of the stored spleens, lymph nodes, lungs, livers, kidneys, and hearts were obtained by frozen section, and the obtained samples were mounted by using a mounting medium including 4′, 6-diamidino-2-phenylindole (DAPI) (Vector Laboratories, Inc.). The knee joint tissues were fixed by incubating the tissues by using 4% (w/v) paraformaldehyd (PFA) (Sigma Aldrich)/PBS (pH 7.4) at 4° C. overnight. Then, the knee joint tissues were decalcified by incubating the tissues by using 5%(w/v) ethylenediaminetetraacetic acid (EDTA) (Sigma Aldrich)/PBS (pH 7.4) (Biosesang Inc.) at 4° C. for about 21 days. The decalcified knee joint tissues sequentially dehydrated in 10%, 20%, and 30% sucrose (Sigma Aldrich)/PBS solution at 4° C. three hours in each solution. Then, the resulting tissues were embedded in an O.C.T. composition (Sakura Finetek). The CM-Dil-labeled stem cells included in the tissue slice samples were observed by using a laser scanning confocal microscope LSM700 (Carl Zeiss), and the results are shown in FIG. 5.

As shown in FIG. 5, in the mice in which rheumatoid arthritis was induced, many hASCs were found in the spleens, and some hASCs were also found in the lungs, lymph nodes, livers, kidneys, and knee joints. In addition, in the mice in which rheumatoid arthritis was induced, CTLA4Ig-hASCs were found in the spleen, lymph nodes, lungs, livers, kidneys, and knee joint-containing bone samples. In normal mice, on the other hand, many hASCs were found in the lungs, and some hASCs were also found in the spleens and livers, but not in the knee joint-containing bone samples.

As described above, according to the one or more of the above embodiments of the present invention, MSCs expressing a fusion protein including a first polypeptide including CTLA4 or a fragment thereof and a second polypeptide including an immunoglobulin constant region are administered to joints, which are the regions affected by arthritis, so that the fusion protein including CTLA4 may efficiently act. The fusion protein including CTLA4 is continuously expressed from the MSCs to act in vivo for an extended period of time, provides a synergic effect with the immune control capacity of MSCs, and provides excellent cartilage-protecting efficacy. Therefore, the pharmaceutical composition for prevention or treatment of arthritis, wherein the pharmaceutical composition includes mesenchymal stem cells expressing a fusion protein including a first polypeptide including CTLA4 or a fragment thereof and a second polypeptide including an immunoglobulin constant region; the kit including an expression vector comprising a first polynucleotide encoding CTLA4 or a fragment thereof and a second polynucleotide encoding an immunoglobulin constant region; and a mesenchymal stem cell; and the method of preventing or treating arthritis by using the same have an excellent effect on the prevention and treatment of arthritis.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

1. A pharmaceutical composition for prevention or treatment of arthritis, wherein the pharmaceutical composition comprises a mesenchymal stem cell (MSC) expressing a fusion protein comprising a first polypeptide including cytotoxic T-lymphocyte antigen 4 (CTLA4) or a fragment thereof and a second polypeptide including an immunoglobulin constant region.

2. The pharmaceutical composition of claim 1, wherein the CTLA4 or the fragment thereof comprises a CTLA4 extracellular domain.

3. The pharmaceutical composition of claim 2, wherein the CTLA4 extra-cellular domain is encoded by a polynucleotide sequence of SEQ ID NO: 2.

4. The pharmaceutical composition of claim 1, wherein the immunoglobulin constant region comprises an Fc (fragment crystallizable) region.

5. The pharmaceutical composition of claim 4, wherein the immunoglobulin constant region is an immunoglobulin γ1 constant region.

6. The pharmaceutical composition of claim 1, wherein the immunoglobulin constant region is encoded by a polynucleotide sequence of SEQ ID NO: 3.

7. The pharmaceutical composition of claim 1, wherein the mesenchymal stem cell is an adipose tissue-derived mesenchymal stem cell.

8. The pharmaceutical composition of claim 1, wherein the mesenchymal stem cellis a human mesenchymal stem cell.

9. The pharmaceutical composition of claim 1, wherein the fusion protein is secreted from the mesenchymal stem cell.

10. The pharmaceutical composition of claim 1, wherein the fusion protein is further fused with a signal peptide at an N-terminal of the fusion protein.

11. The pharmaceutical composition of claim 10, wherein the signal peptide is encoded by a polynucleotide sequence of SEQ ID NO: 1.

12. The pharmaceutical composition of claim 1, wherein the arthritis is rheumatoid arthritis.

13. A kit for prevention or treatment of arthritis, wherein the kit comprises:
an expression vector comprising a first polynucleotide encoding cytotoxic T-lymphocyte antigen 4 (CTLA4) or a fragment thereof and a second polynucleotide encoding an immunoglobulin constant region; and
a mesenchymal stem cell.

14. The kit of claim 13, wherein the first polynucleotide comprises a polynucleotide sequence of SEQ ID NO: 2.

15. The kit of claim 13, wherein the second polynucleotide comprises a polynucleotide sequence of SEQ ID NO: 3.

16. The kit of claim 13, wherein the expression vector further comprises a polynucleotide sequence of SEQ ID NO: 1.

17. A method of preventing or treating arthritis, wherein the method comprises administrating an MSC expressing a fusion protein comprising a first polypeptide including cytotoxic T-lymphocyte antigen 4 (CTLA4) or a fragment thereof and a second polypeptide including an immunoglobulin constant region to a subject.

18. The method of claim 17, wherein the administration is a parenteral administration.

19. The method of claim 17, wherein the subject is one mammal selected from the group consisting of a human, a dog, a cat, a pig, a horse, cattle, sheep, a mouse, and a monkey.

20. The method of claim 17, wherein the arthritis is rheumatoid arthritis.


SEQUENCE LISTING (4 sequences)

SEQ ID NO: 1 - DNA, 75 bp, Artificial Sequence
  polynucleotide encoding human oncostatin M signal peptide
atgggggtactgctcacacagaggacgctgctcagtctggtccttgcactcctgtttccaagcatggcgagcatg

SEQ ID NO: 2 - DNA, 372 bp, Artificial Sequence
  polynucleotide encoding mouse CTLA4 extracellular domain
atacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgac

SEQ ID NO: 3 - DNA, 684 bp, Artificial Sequence
  polynucleotide encoding hinge-CH2-CH3 region of human immunoglobulin gamma 1 constant region
gtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 4 - DNA, 1162 bp, Artificial Sequence
  target polynucleotide
ggatcccaccatgggggtactgctcacacagaggacgctgctcagtctggtccttgcactcctgtttccaagcatggcgagcatgtctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacgaattcgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgactcgag